(19) United States
(12) Patent Application Publication
(10) Pub. No.: US 2009/0137022 A1
(43) Pub. Date: May 28, 2009

(54) Extracting and purifying beta 1,4-xylanase

(75) Inventors: Michael Patane, Seaforth (AU); Mitchell Parker, Seaforth (AU); Akshat Talwalkar, Seaforth (AU)

Correspondence Address: Ballard Spahr Andrews & Ingersoll, LLP, SUITE 1000, 999 PEACHTREE STREET, ATLANTA, GA, 30309-3915, US

(73) Assignee: PROTECH RESEARCH PTY LTD, SEAFORTH, NEW SOUTH WALES (AU)

(21) Appl. No.: 11/991,096

(22) PCT Filed: Aug. 25, 2006
(86) PCT No.: PCT/AU2006/001241

§ 371 Date: Feb. 27, 2008

(30) Foreign Application Priority Data
Mar. 8, 2005 (AU) 2005904734

(51) Int. Cl. C12N 9/26

(52) U.S. Cl. 435/201

(57) ABSTRACT

The invention relates to extracting and purifying an enzyme from a cell, particularly, but not exclusively, to extracting and purifying a β1,4-xylanase.

### FIELD OF THE INVENTION

The invention relates to extracting and purifying an enzyme from a cell, particularly, but not exclusively, to extracting and purifying a β1,4-xylanase.

### BACKGROUND OF THE INVENTION

β1,4-xylanase (EC 3.2.1.8), otherwise known as 1,4-β-xylan xylanohydrolase; endo-1,4-β-xylanase; endo-(1→4)-β-xylanase(1→4)-β-xylan 4-xylanohydrolase; endo-1,4-xylanase; xylanase; β-1,4-xylanase; endo-1,4-xylanase; endo-β-1,4-xylanase; endo-1,4-β-D-xylanase; 1,4-β-xylan xylanohydrolase; β-xylanase; β-1,4-xylan xylanohydrolase; endo-1,4-β-xylanase; or β-D-xylanase, is an enzyme that catalyses the endohydrolysis of 1,4-β-D-xylosidic linkages in xylans.

β1,4-xylanase is particularly important in food industries.

The processes for obtaining commercial quantities of β1,4-xylanase tend to be difficult to operate on a commercial scale, in terms of requiring sophisticated fermentation technology, extraction and separation techniques, multiple steps and expensive reagents and equipment. Some processes are characterised by an unacceptable loss or wastage of β1,4-xylanase. Other processes tend to produce a non purified final product that has a sub-optimal specific activity.

In view of the above, there is a need for improved processes for purification of β1,4-xylanase.

### SUMMARY OF THE INVENTION

The invention seeks to at least minimise one or more of the above identified problems or limitations and/or to provide an improved process for purification of β1,4-xylanase.

In one aspect, the invention provides a process for purifying β1,4-xylanase from a cell. The process includes the step of heating an extract of a cell formed from a solution including at least one divalent cation, to increase the specific activity of β1,4-xylanase in the extract.

In another aspect, the invention provides a process for purifying β1,4-xylanase from a barley cell. The process includes the following steps:

(a) releasing β1,4-xylanase from a barley cell into a solution including Calcium and Magnesium to form an extract;

(b) heating the extract to increase the specific activity of 1,4-xylanase in the extract.

In another aspect, the invention provides a process for purifying β1,4-xylanase from a barley cell. The process includes the following steps:

(a) releasing β1,4-xylanase from a barley cell into a solution including Calcium and Magnesium to form an extract;

(b) heating the extract to increase the specific activity of β1,4-xylanase in the extract; and

(c) utilising anion exchange chromatography to purify β1,4-xylanase from the heated extract.

Typically the cell is a barley cell, such as a cell derived from a barley grain or barley rootlet.

In another aspect, the invention provides β1,4-xylanase produced by the process of the invention.

In another aspect, the invention provides a cell including β1,4-xylanase produced by the process of the invention.

### DETAILED DESCRIPTION OF THE EMBODIMENTS

As described herein, the inventor has found that creating a suitable buffered environment during extraction and conducting heat treatment of an extract of a barley cell or rootlet in a solution comprising at least one divalent cation permits the specific activity of the extract with respect to β1,4-xylanase contained within it to be increased. For example, the specific activity of a heat treated extract of a barley rootlet formed from a solution comprising 50 mM Calcium Chloride and 50 mM Magnesium Chloride was observed to increase 2.4 fold over a non heat treated sample (709 μmoles/min/mL compared with 287.85 μmoles/min/mL). Further, a heat treated extract containing 50 mM Calcium Chloride and 50 mM Magnesium Chloride was observed to have an improved specific activity (356.5 μmoles/min/mL) compared with a heat treated extract containing no Calcium and Magnesium (56.1 μmoles/min/mL).

This is a significant finding because it permits heat treatment, a purification step that is relatively simple to operate on a commercial scale, to be implemented with minimal loss of activity of β1,4-xylanase.

Thus in certain embodiments there is provided a process for purifying a β1,4-xylanase from a cell including the step of heating an extract of a cell formed from a solution including at least one divalent cation, to increase the specific activity of a β1,4-xylanase in the extract.

In other embodiments there is provided a process for increasing the specific activity of a β1,4-xylanase in a cell extract, said extract being one formed from a solution including at least one divalent cation. The process includes the step of heating the cell extract to increase the specific activity of a β1,4-xylanase in the extract.

It is believed that the specific activity of the extract is increased because the divalent cation protects β1,4-xylanase from denaturation at temperatures at which other proteins in the extract are degraded.

Typically, the at least one divalent cation in the solution may be Calcium and/or Magnesium. For example, the solution may contain Calcium Chloride and/or Magnesium Chloride.

Zinc, copper and manganese are also cations.

The Calcium and Magnesium ions may be included in the extract in an amount to permit control of the denaturation of β1,4-xylanase when the extract is heated. Typically, Calcium and Magnesium are included in the extract in an amount to at least limit the denaturation of β1,4-xylanase when the extract is heated. For example, the concentration of Calcium may be less than 100 mM and the concentration of Magnesium may be less than 100 mM.

A concentration of Calcium and Magnesium in a range between about 25 to 50 mM is particularly useful as further down stream processing of the extract for further purification, such as anion exchange chromatography, may require removal of Calcium and Magnesium. Accordingly a concentration of Calcium ions of about 50 mM and a concentration of Magnesium ions of about 50 mM is particularly useful.

In certain embodiments, the Calcium ions are provided in a concentration selected from the group consisting of 10 mM, 15 mM, 20 mM, 25 mM, 30 mM, 35 mM, 40 mM and 45 mM.

In certain embodiments, the Magnesium ions are provided in a concentration selected from the group consisting of 10 mM, 15 mM, 20 mM, 25 mM, 30 mM, 35 mM, 40 mM and 45 mM.

Further, the solution may be buffered to between about pH 7 and 9 using a suitable solution, such as Tris HCl. In particular, and with reference to the preceding, it has been found that by heating an extract buffered at pH 8.9 with 200 mM TrisHCl and containing divalent cations, the specific activity of the extract can be increased at least 6.35 fold (356.5 μmoles/min/mL) compared with a heat treated extract containing no Calcium or Magnesium (56.1 μmoles/min/mL).

Thus in certain embodiments, there is provided a process for purifying a β1,4-xylanase from a cell. The process includes a step of heating an extract of a cell formed from a solution having a pH of at least about 7 and including at least one divalent cation, to increase the specific activity of a β1,4-xylanase in the extract.

Typically the solution has a pH of at least about 8, although higher ranges to about pH 9.0 are particularly useful for enhancing the specific activity of the enzyme in the extract. Examples of suitable pH conditions include 7.5, 8.0, 8.5, 9.0. A pH of above 9.0 could be used, however above this range the activity of the enzyme tends to be affected. Trizma base buffered in HCl in a concentration of about 200 mM is particularly useful to provide the appropriate pH.

It is believed that major constituents of an extract of a barley cell include a number of enzymes having activity for various carbohydrate and protein substrates. Thus, the extract is typically heated to a temperature that permits denaturation of unwanted proteases, ancillary enzymes, or otherwise, destruction of activity of these enzymes in the extract. As described herein, temperatures less than 70° C. are suitable for this purpose.

It is particularly advantageous to heat the extract to between about 45 and 65° C. because at temperatures approaching 70° C. and above, β1,4-xylanase activity may be lost. Accordingly, a temperature of about 60° C. is particularly useful.

The inventor has also found that the purification of β1,4-xylanase from a barley cell extract can be improved by extracting a barley cell homogenate at 40° C. in a solution including Calcium and Magnesium. Specifically, as described herein, the specific activity of an extract comprising Calcium and Magnesium after maintenance at 40° C. was found to be 363.8 μmoles/min/mL as compared with the activity of an extract maintained at 40° C. in the absence of Calcium and Magnesium (61.3 μmoles/min/mL).

It is believed that maintenance of such an extract at 40 C is important because it permits β1,4-xylanase to disassociate from solids in the extract, and accordingly, to solubilise into the liquid phase of the extract, prior to further processing of the extract, such as a heat treatment step or a chromatographic separation step. The Calcium and Magnesium are believed to be important for limiting hydrolysis of the enzyme during the maintenance of the extract at 40° C. Thus in accordance with the invention, a process for purifying β1,4-xylanase from a barley cell includes the following steps:

(a) releasing β1,4-xylanase from a barley cell into a solution including Calcium and Magnesium to form an extract; and

(b) heating the extract to increase the specific activity of β1,4-xylanase in the extract.

Typically, the extract is maintained in conditions for promoting stabilization of the β1,4-xylanase in the extract prior to heating the extract.

The extract may be maintained at less than 10° C. for less than 3 days. For example, the extract may be maintained between 0 to about 4° C. for between about 1 to 48 hours.

It is particularly advantageous to maintain the extract for 12 hours at 4° C. prior to extraction at 40° C. as this improves the speed of purification protocols that comprise further purification steps.

The inventor has been further found that β1,4-xylanase can be purified to virtual homogeneity from a barley cell extract by a process including the following steps:

(a) releasing β1,4-xylanase from a barley cell into a solution including Calcium and Magnesium at pH of about 8.9 to form an extract;

(b) heating the extract to increase the specific activity of β1,4-xylanase in the extract; and

(c) utilising chromatography to purify β1,4-xylanase from the heated extract.

As described herein, β1,4-xylanase can be further purified from a heat treated barley cell extract by anion exchange chromatography. Accordingly, typically, in step (c), anion exchange chromatography is utilised to purify β1,4-xylanase from the heated extract.

The inventor has found that Calcium and Magnesium ions tend to limit binding of β1,4-xylanase during anion exchange chromatography. Accordingly, typically the extract is desalted before anion exchange chromatography. One way of desalting to remove Calcium and Magnesium ions is by ultrafiltration-diafiltration. Alternatively, a preparative de-salting column, such as a Hi Prep 26/10 desalting column can be used. It is particularly advantageous to remove substantially all of the Calcium and Magnesium from the extract prior to anion exchange chromatography for the purpose of maximising the yield of β1,4-xylanase purified from the anion exchange column.

Typically, the extract is maintained in conditions for promoting solubilisation of the β1,4-xylanase in the extract prior to heating the extract.

In the processes of the invention described above, the extract of the barley cell is typically produced by homogenising barley cells in an appropriate buffer. One way of homogenizing grains is by use of a blender, such as a Waring blender. Alternatively, the extract may be produced by milling barley grains in an appropriate buffer using a roller mill following a predetermined steeping and germination protocol to enhance enzyme extraction.

The solution into which the β1,4-xylanase from the cell is released to form an extract is typically a buffer for controlling pH. Solutions prepared from Trisma base are examples of such a solution. A solution having a concentration of no more than about 300 mM Tris is suitable, for example, 200 mM Tris is particularly advantageous adjusted and maintained at a pH 8.9.

It will be understood that the processes of the invention are useful for purifying β1,4-xylanase from cells other than barley cells. Other examples include cells of grains such as rice and wheat, legumes, pulses and other vegetable matter. Further, it will be understood that processes of the invention are useful for isolating barley β1,4-xylanase from cells that contain a recombinant nucleic acid molecule that encodes barley β1,4-xylanase. Examples of such cells include bacterial cells and yeast cells.

### EXAMPLE 1

### Materials and Equipment

Germinating barley seeds (Schooner variety) were obtained from Barrett Burston Malting, (Thornleigh, NSW, Australia), Calcium Chloride, Magnesium Chloride, Potassium Chloride, Sodium Chloride, Trisma base, Sodium Acetate and Hydrochloric Acid were supplied by Sigma Aldrich (Castle Hill, NSW, Australia), azo-wheat Arabinoxylan test kit was obtained from Megazyme ( Bray, Ireland) and undenatured Ethanol was purchased from CSR Distilleries (Ingleburn, NSW, Australia).

The germinated barley grains were milled on a Kustnel Freres & Cie roller mill to a gap setting of 1 mm to crack the grains allowing extraction of enzymes.

The crude enzyme extract was coarse filtered though double cheesecloth then centrifuged at 26,800×g for 30 minutes at 4° C. to remove any precipitate.

The crude extract was concentrated and buffer exchanged using a MidGee cross flow ultrafiltration unit combining a Masterflex economy drive peristaltic pump and Masterflex Easy load II head, UFP-10-C-H42LA ultrafiltration cartridge with 10 kDa nominal cut off and MidGee starter kit KMDG-1. A flow rate of 17 mL per minute at 10 psi pressure was sufficient to separate and concentrate the β1,4-xylanase containing fractions.

The buffer used for FPLC gel filtration and ion exchange chromatography was 25 mM Sodium Acetate (pH 5.5 ). The eluent buffer for ion exchange chromatography included 1 M NaCl.

An Amersham Pharmacia AKAT gradient processing FPLC system complete with a 900 model monitor, lamp and detector (set at 280 nm), 920 model pump and Frac 950 fraction collector interfaced to a Compaq Deskpro Pentium III computer supporting Unicorn analytical software was used for all protein purification. The columns used included a Hi Prep 26/10 desalting column connected to a Super loop 50 (to facilitate larger injection volumes), a 16/10 Hi-Prep DEAE FF anion exchange column with a final purification undertaken on a Mono Q HR 5/5 column.

Isolation of β1,4-xylanase was identified by the presence of single protein bands on native electrophoresis gels and single absorption peaks by sequential anion exchange chromatography.

An LW Scientific UV-Visible spectrophotometer was used to measure enzyme activity operating at 590 nm. The system was controlled by a Celeron processor computer operating a LW Scientific Graphite version 3.1 enzyme kinetics software program.

### EXAMPLE 2

### Preparation of a Standard Curve for Dye Labelled Wheat Arabinoxylan to Determine β1,4-xylanase Activity

A standard curve for the identification of β1,4-xylanase activity was supplied by Megazyme utilising a Azo-Wheat Arabinoxylan substrate. The Arabinoxylan substrate is prepared by dyeing highly purified and partially depolymerised wheat Arabinoxylan with Remazobrilliant Blue dye. The Azo-Wheat Arabinoxylan substrate (1.0 g) is added to 100 mL of boiling water and vigorously stirred. The solution is cooled to room temperature and the volume readjusted to 100 mL. Sodium azide (0.02g) is added as a preservative and the solution is stored at 4° C. Working standards are prepared in the range of 20 to 560 μM/mL in 100 mM Sodium Acetate buffer at pH 4.5 and read spectrophotometrically at 590 nm.

### EXAMPLE 3

### Preparation of a Standard Curve for Protein to Determine β1.4-xylanase Specific Activity

Protein was determined using the BioRad micro assay procedure derived from the original method of Bradford utilising a standard curve produced for bovine serum albumin. Each analysis was conducted in duplicate requiring incubation at room temperature for 10 minutes with the absorbance measured at 595 nm. Standards were prepared in the range of 0.2 to 1.4 mg/mL of protein.

### EXAMPLE 4

### Enzyme Kinetics Assay

The assay requires 0.5 ml of the extracted enzyme solution (post buffer exchange) pre-equilibrated at 40° C. for 10 minutes. To this suspension is added 0.5 mL of the pre equilibrated substrate solution [1.0 g in 100 mL of boiling water]. The mixture is stirred and incubated at 40° C. for exactly 10 minutes. The reaction is terminated by the addition of 2.5 ml of 95% (v/v) ethanol vortexing for 10 seconds. The reaction tubes are allowed to equilibrate at room temperature for 10 minutes and then centrifuged at 1,500×g for 10 minutes to precipitate the higher molecular weight fractions of the substrate. The supernatant is transferred directly to a curvette and the absorbance read at 590 nm. Activity is determined by reference to the standard curve.

### EXAMPLE 6

### Preparation of Crude β1,4-xylanase Extract

30 g of 3 to 12 month old stored barley grains were dispersed in 45 mL 0.2M Tris-HCl (pH 8.9) containing 50 mM MgCl₂ and 50 mM CaCl₂ following a germination period. The germinated grains were firstly milled using smooth rollers at a gap setting of 1 mm and speed 440 rpm, feed rate of 1 kg per minute prior to extraction at 40° C. for 2 hours to facilitate solubilisation of β1,4-xylanase.

The insoluble material was removed from the extract by filtering through double cheese cloth. The filtrate was centrifuged at 15,000 rpm for 30 minutes at 4° C. to remove solids and the supernatant was passed through a 0.45 μM filter and stored at 4° C. in a sterile container with 0.01% sodium azide. This process formed the crude β1,4-xylanase extract.

The activity and specific activity of the crude β1,4-xylanase extract was then determined according to Examples 2, 3 and 4 above.

### EXAMPLE 7

### Purification of β1,4-xylanase from the Crude Extract

The first stage of the purification process involved the removal of heat labile proteases, inhibitory proteins and any superfluous proteinaceous materials from the crude β1,4-xylanase extract with the aim of reducing any loss of activity or damage to the structure of β1,4-xylanase while increasing the specific activity of β1,4-xylanase extract. To inactivate and remove these proteins, the crude extract was heated in a water bath to 60° C. and maintained at that temperature for 1 hour. The extract was then cooled to room temperature and buffer exchanged by cross flow ultrafiltration-diafiltration with 25 mM Sodium Acetate pH 5.5 to facilitate gel filtration and ion exchange chromatography. The extract was initially centrifuged and filtered through a 0.45 μm filter.

The activity and specific activity of the heat treated β1,4-xylanase extract was then determined according to Examples 2, 3 and 4 above.

Gel filtration and ion exchange chromatography was then undertaken. A 50 mL sample of the extract was injected into a Super loop 50 column and gel filtered by FPLC on a Hi Prep 26/10 desalting column at a flow rate of 7.0 mL per minute, to remove magnesium and calcium The desalted fractions were then pooled and reloaded onto the Super loop column and passed through a Hi PREP 16/10 DEAE anion exchange column at 3.0 mL per minute to initially fractionate β1,4-xylanase. The isolated fraction was again desalted to remove the 1M NaCl elution buffer and purified by passing the fraction through the Mono Q HR 5/5 column at 1.5 mL per minute. A single peak was obtained and analysed for activity and specific activity according to Examples 2 to 4 above.

### EXAMPLE 8

### Purification Profile for β1,4-xylanase

The results for the purification of β1,4-xylanase are shown in Table 1.

TABLE 1

| Sample | Activity | Specific activity | Purification factor |
|---|---|---|---|
| Crude extract | 380 | 287.85 | 1 |
| Heat treatment | 390 | 709 | 2.46 |
| Anion exchange | 392.5 | 8451.27 | 29.36 |

Activity: ×10³ μmoles/min/mL
Specific activity: ×10³ μmoles/min/mL/mg

### EXAMPLE 9

### Effect of Calcium, Magnesium on Limit β1,4-xylanase Activity of Crude Extract During Solubilization at 40° C.

We sought to determine whether calcium magnesium would have an effect on stabilisation of β1,4-xylanase in the crude extract, or otherwise, on preserving or enhancing β1,4-xylanase activity of the crude extract, during the step of extracting β1,4-xylanase at 40° C. that follows the milling step described in Example 6.

To this end we extracted the enzyme in (i) water, (ii) 0.2M Tris-HCL (pH 8.9), (iii) 0.2M Tris-HCL (pH 8.9) with 50 mm Calcium Chloride and 50 mm Magnesium Chloride maintained the extract at 40° C. for 2 hours. We found that the buffer containing 0.2M Tris-HCL maintained at a pH of 8.9 with the addition of 50 mM Calcium Chloride and 5 mM Magnesium Chloride enhanced and indeed stabilised 1,4-xylanase activity compared to water (observed over a decreasing range of pH). The β1,4-xylanase activity was 590% greater than in the sample with no Calcium, Magnesium or L-Cysteine at decreasing pH, (363.8 μmoles/min/mL compared to 61.3 μmoles/min/mL).

1. A process for purifying a β1,4-xylanase from a cell including the step of heating an extract of a cell formed from a solution including at least one divalent cation, to increase the specific activity of β1,4-xylanase in the extract.

2. The process according to claim 1 wherein the divalent cation is calcium or magnesium.

3. The process according to claim 1 wherein the divalent cation is calcium and magnesium.

4. The process according to claim 1 wherein the divalent cation is provided in the solution in a concentration of less than about 100 mM.

5. The process according to claim 1 wherein the solution has a pH of at least about 5.

6. The process according to claim 5 wherein the solution has a pH of about 8.9.

7. The process according to claim 1 wherein the solution is heated to less than about 70° C.

8. A process according to claim 1 wherein the cell is a barley cell.
